(19) United States
(12) Patent Application Publication
(10) Pub. No.: US 2002/0038123 A1
(43) Pub. Date: Mar. 28, 2002

(54) Osteotomy implant

(76) Inventors: Jeffrey L. Visotsky, Des Plains, IL (US); Jeffrey L. Halbrecht, San Francisco, CA (US)

Correspondence Address: Bencen & Van Dyke, P.A., 1630 Hillcrest Street, Orlando, FL, 32803, US

(21) Appl. No.: 09/873,942

(22) Filed: Jun. 4, 2001

Related U.S. Application Data
(63) Continuation of application No. 09/665,716, filed on Sep. 20, 2000, now abandoned.

(51) Int. Cl. A61B 17/58

(52) U.S. Cl. 606/73

(57) ABSTRACT

A tapered screw or dowel made from allograft, autograft or xenograft bone is used to keep an osteotomy or fracture distracted during healing to result in proper alignment of the knee, wrist or any other skeletal site where an opening osteotomy is required to provide for improved bone alignment.

### FIELD OF THE INVENTION

The invention provides a bone screw or dowel made of allograft bone, for use in an osteotomy or fracture to result in proper bone alignment.

### BACKGROUND INFORMATION

Various devices and methods have been developed to assist in the correction of a malaligned joint or in the repair of a fracture. Malaligned knees have been corrected according to methods known in the art using either an opening or closing wedge osteotomy. A closing wedge osteotomy requires the surgeon to cut a wedge out of the bone and hold the bones together with a metallic fracture plate and screws or other external fixation device. An opening wedge osteotomy requires a surgeon to create a fracture in a bone into which a wedge is inserted and held in place with metallic plates and screws or external fixators. Distal radial fractures are traditionally fixed with metallic pins, wires, and screws, which require the surgeon to “eyeball” the proper alignment.

In U.S. Pat. No. 5,766,251, a wedge-shaped spacer usable for varus, valgus, flexion, extension, and derotation osteotomies is disclosed. The spacer is made of sintered hydroxyapatite and contains at least one thorn-shaped projection extending outwardly from the upper or lower surface and one hole extending from the upper surface to the lower surface. The sintered hydroxyapatite lacks the collagen fibers found in bone, therefore the hydroxyapatite is not analogous to the allograft bone used in the present invention. In addition, the wedge-shaped spacer requires the use of plates and screws to be held in place in the osteotomized site. While both the wedge-shaped spacer of the '251 patent and the present invention are used for corrective osteotomies, the '251 patent does not teach or suggest the novel device and method of the present invention.

In U.S. Pat. No. 6,008,433, an osteotomy device, kit and methods for realigning varus angulated knees is disclosed. The device is similar to the '251 patent in that it is substantially wedge-shaped. The '433 patent does not disclose a device made from bone. In addition, the '433 patent does not have a substantially circular cross section which is embodied in the present invention. Other than disclosing a device and method for realigning varus angulated knees, the '433 patent does not teach or suggest the novel device and method of the present invention.

For other devices and methods developed to assist in the correction of malaligned joints, or in the repair of a radial fracture see U.S. Pat. No.'s 5,868,749; 5,968,047; 5,180,382.

Accordingly, having reviewed devices and methods known in the art, it is concluded that there remains a need for an osteotomy device that potentially eliminates the need for metallic plates and screws or other external fixation devices. The present invention provides such a device.

### BRIEF SUMMARY OF THE INVENTION

A tapered screw or dowel made from allograft, autograft or xenograft (cortical or cancellous or both) bone or combinations thereof is used to keep an osteotomy or fracture distracted during healing to result in proper alignment of the knee, wrist or any other skeletal site where an opening osteotomy is required to provide for improved bone alignment. In one embodiment, the implant of this invention has a smaller diameter front end which is flat, and a larger diameter back end which is provided with a notch, slot, circular, hexagonal or other shaped protrusion or indenture, cannulation or other structures known in the art enable the use of an insertion device for implantation. The larger diameter end of the implant preferably includes a cannular opening to allow for either a guide or insertion device in positioning the implant into the fracture, or for use as a means to secure the implant onto an insertion device during implantation. The implant is preferably provided with an opening to be filled with autogenic bone, allogenic bone, a demineralized bone product or other osteogenic factors, cells or the like to stimulate healing in the fracture.

The implant is inserted into the fracture after the fracture is opened with a distracter to accommodate the smaller diameter front end. The distracter is later removed from the surgery site. Threads on the implant allow it to be screwed in to control the angle of distraction. In addition, threads ensure that the implant remains in place inside the fracture. The remaining portion of the implant, which is not screwed into the fracture site, can be removed using a saggital saw or similar device. A larger implant can be used for large bone osteotomies, such as tibial or femoral osteotomies. A smaller version can be used for small bone osteotomies, such as in the distal radius.

Accordingly, it is one object of this invention to provide an osteotomy device made from bone, a bone-like substance, or a biocompatible synthetic material for use in an osteotomy procedure.

Another object of this invention is to provide proper alignment of bones, whether after an osteotomy or trauma, without the need for plates and screws, or wires and pins to hold the implant in place.

Another object of this invention is to provide an implant with at least one cavity, and preferably a plurality of microcavities (specify a size range?, and define a microcavity?) running therethrough to accept packing having osteogenic properties.

Another object of this invention is to provide an implant with improved osteogenic and bone fusion-promoting capacity.

Another object of this invention is to provide a method for using the novel osteotomy implant of this invention.

Another object of this invention is to provide a method for making the novel osteotomy implant of this invention.

Additional objects and advantages of the osteotomy implant of this invention will become apparent from a review of the full disclosure which follows.

### BRIEF DESCRIPTION OF THE DRAWINGS

FIG. 1 depicts the structure of one embodiment of the osteotomy implant.

FIG. 2A is a top view of the osteotomy implant depicted in FIG. 1 showing a slot opening running therethrough.

FIG. 2B is a back view of the osteotomy implant depicted in FIG. 1 showing a cannulation and notch used for insertion of the device.

FIG. 3 is a top view of one embodiment of the implant depicted in FIG. 1 showing holes radiating from the canal opening.

FIG. 4A is a side view of the osteotomy implant depicted in FIG. 1 showing a side view of the notch used for insertion of the implant.

FIG. 4B is a magnified view of the element shown as “A” in FIG. 4A.

### DETAILED DESCRIPTION OF THE PREFERRED EMBODIMENTS

The implant of this invention is useful in the elongation or realignment of bones by means of creating an intentional fracture, such as in an osteotomy, or in the event of a trauma. Elongation or realignment of bones is necessary to prevent and correct such problems as osteoarthritis or varus and valgus angulated knees. A varus angulated knee condition is commonly referred to as “bowleg”, and a valgus angulated knee condition is commonly referred to as “knock-knee”.

In an osteotomy, a surgeon intentionally creates a fracture, or transverse incision in a bone, distracts the bone incision, and inserts an implant, wires or pins, which allow the bones to be distracted during healing. Common areas for use of the present invention include the proximal tibia, distal femur and distal radius, but the implant can be used in other bones where realignment is necessary. Unlike other implants and devices known in the art, the present invention does not require, or reduces the need for, plates and screws or other external fixation devices to keep the implant in place.

In order to make the implant of the present invention, bone banks recover allograft bone from donor bone, whether autologous, allograft (e.g. from a human cadaver) or xenograft, (e.g. from an animal cadaver). The donor must be screened for communicable diseases, cancer, and at-risk behavior prior to bone donation. The bone pieces obtained from a donor can then be divided into blanks using an oscillating bone saw. The surfaces of the bone blanks are preferably planed smooth, for example, using a diamond plane under cool water. The bone blanks are then machined into the form of cylinders. One skilled in the art may refer, for example, to methods disclosed in U.S. Pat. No. 5,814,084, hereby incorporated by reference for this purpose. The bone blank cylinder is then machined in a lathe or equivalent device to produce, for example, a conical shape with a smaller diameter front end and a larger diameter back end. A lathe can also be used to inscribe threads, grooves or other external features into the circumference of the bone blank. At least one slot or cavity is optionally machined into the body of the cylinder to allow for biocompatible packing material to be inserted therein prior to implantation. Holes radiating from the at least one slot to the exterior of the implant may also optionally be drilled or formed by means of laser or other means, to permit diffusion of osteogenic materials from the central portions of the implant toward the external portions of the implant, and to permit recipient cells to migrate into the implant to effect expedited remodeling of the implant into host bone. An instrument attachment cannulation may optionally be machined into the back end of the implant by such methods as drilling and/or tapping. The implant is further, optionally, provided with a notch on the back end of the implant for use as a means to drive the implant into the fracture site during implantation and as an orientation marker. The implant may be autoclaved for thermal disinfection, or other disinfection means known in the art. One preferred method hereby incorporated by reference, is the method disclosed in publication number WO 00/29037, hereby incorporated herein by reference. According to that methodology, various cleaning solutions are used to achieve deep interpenetration, cleaning and decellularization of the implant by enclosing the implant in a sealed chamber in the presence of the cleaning solution, and rapidly cycling the pressure within the chamber. The same methodology may be employed to infuse desired biologically active substances into the interior and interstices of the implant, such as growth factors, bone morphogenetic proteins, nucleic acids, antibiotics, anti-inflammatory substances, and the like.

Specific dimensions of the implant are provided below, but those skilled in the art will recognize that these specifics may be appropriately scaled, depending on the size implant required for a given application.

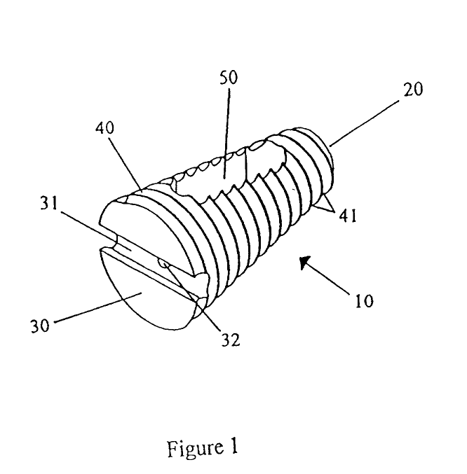

FIG. 1 depicts a perspective view of one embodiment of the novel osteotomy implant 10. The implant is substantially conical in shape and made substantially of bone, a bone-like substance, or a biocompatible synthetic material. The implant has a front end 20 and a back end 30, with the back end 30 having a larger diameter than the front end 20. The back end 30 comprises a notch 31 for receiving a means to drive said implant into the fracture site. Back end 30 also contains a center cannulation 32 set inside notch 31 for use either for a guide wire to position the implant into the fracture site or for use as a means to secure the implant in or onto an insertion device during implantation The cannulation 32 can run from the back end 30 to the front end 20 in order to be used for a guide wire or run partially through the back end 30 for means to attach the implant to an insertion device. An appropriate insertion device as is known in the art for implantation of this type of implant can be used. The body 40 of the osteotomy implant 10 can either be threaded, contain grooves or contain barbs. FIG. 1 depicts the osteotomy implant 10 with threads. The threads 41 permit the osteotomy implant 10 to be screwed into a fracture site that has been distracted during surgery. The ability to screw in the implant allows the surgeon to achieve the appropriate angle for correction. The threads 41, also ensure that the implant stays inside the fracture site by itself, which reduces the need to use plates and screws, wires or pins, although use of such retention means in combination with the present implant is not excluded. The remaining end of the implant, which is not inserted into the fracture site, is then cut off by the surgeon using a saggital saw or similar device.

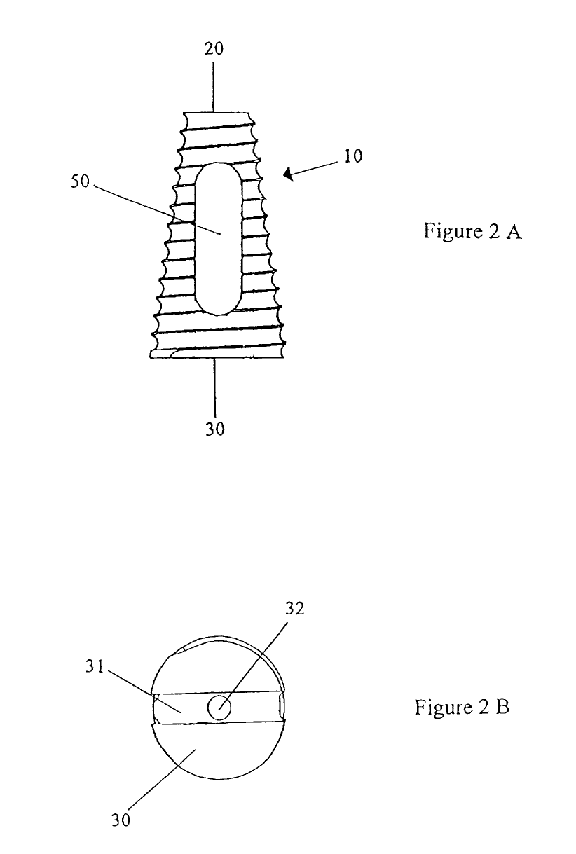

FIG. 2A depicts a top view of the implant 10. The length of the implant 10 from front end 20 to back end 30 can range from about 1 mm to about 60 mm, but is preferably about 15-20 mm in length. The ratio of the back end diameter to the front end diameter ranges from about 5:1 to about 1:1, but preferably has the ratio of about 8:1 wherein the back end 30 diameter is about 19.2 mm and front end 20 diameter is about 6 mm. Slot 50 of implant 10 is formed transversely through the implant. Slot 50 can be filled with autogenous bone, allogenic bone, xenograft bone, demineralized bone, bone paste, cellular material, growth factors, and the like to stimulate healing and remodeling of the implant within the fracture site. The length of the slot 50 can range between about 0.25 mm to about 15 mm, but is preferably about 8-10 mm in length. The width of the slot 50 can range between about 0.1 mm to about 8 mm, but about 3-7 mm is preferred to avoid the walls of the implant 10 from being too thin. It will be apparent to those skilled in the art that the slot 50 may be a plurality of slots. It will also be apparent to those skilled in the art that the size ranges provided here are not limiting but are merely a guide. For example, in radial fractures, very small implants are required.

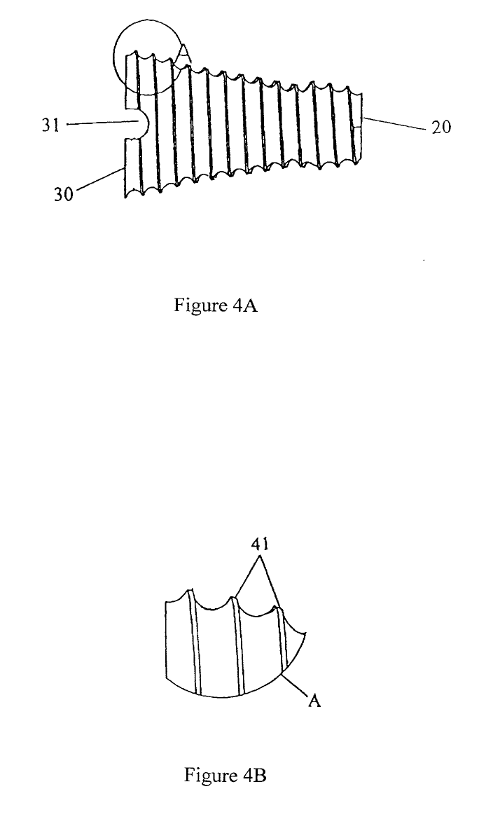

FIG. 2B depicts the back end 30 view of the implant. The width of the notch 31 can range between about 0.1 mm and about 8 mm, but is preferably about 3-5 mm wide. The depth of notch 31 is depicted in FIG. 4A and can range between about 0.1 mm to about 6 mm, but is preferably about 2 mm deep.

The cannulation 32 is depicted in FIG. 2B. The cannulation 32 diameter can vary between about 2 mm to about 4 mm, but is preferably about 2 mm. The length of the cannulation can extend either partially or completely through the length of the implant.

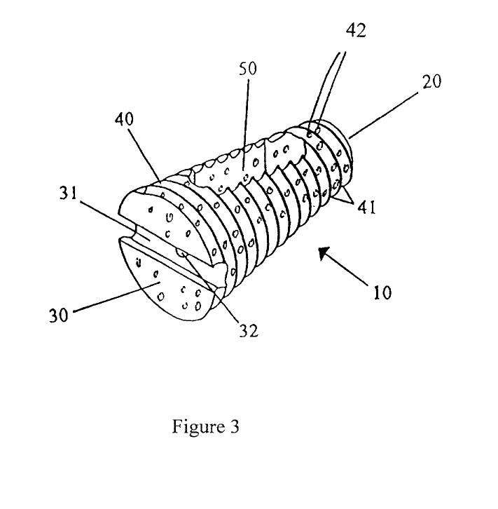

FIG. 3 depicts a top view of one embodiment of the implant containing holes 42 in the implant 10 which further stimulate healing in the fracture by allowing the autogenous bone, allogenic bone, xenograft bone, demineralized bone, bone paste, cellular material, growth factors, and the like, which is placed in the slot opening 50 to pass through the body 40 of the implant 10. The holes 42 can range in size, but are preferably about 200 μm in size to permit the autogenic bone, allogenic bone, xenograft bone, demineralized bone, bone paste, cellular material, growth factors, and the like, to pass through without compromising the strength of the implant 10. Such holes 42 or canals also permit rapid invasion of recipient cells into the implant, and diffusion out of the implant of, for example, mesenchymal stem cells infused or packed into the implant. As a result, the implant is more rapidly remodeled into recipient bone. Further, the implants may be assembled by combining portions of bone from different or the same donor, from allograft bone, autograft bone, xenograft bone, cortical bone, cancellous bone and synthetic materials may also be combined to form an appropriate implant according to this invention. The assembled pieces may be held together by adhesive, by pins (metal cortical bone, synthetic) or other fixation means. Due to the different properties of cortical bone and cancellous bone, a composite assembled implant according to this invention may be made with a wide range of physical, chemical and biological properties.

FIG. 4A provides a side view of the implant shown in FIG. 1. FIG. 4B is a magnified view of the screw threads 41 depicted on the body 40 of the implant in FIG. 4A. The threads extend from the front end 20, to the back end 30 of the implant 10. Pitch can vary between about 0.5 mm to about 3 mm, but is preferably about 1.5 mm from point to point. The threads are relatively perpendicular to the implant body 40.

What is claimed is:

1. An implant having a substantially circular or elliptical cross section, comprising a front end of a first diameter, a rear end of a second diameter, wherein said first diameter is smaller than said second diameter, at least one slot formed transversely through a portion of said implant, and a means for attaching said implant to an implant insertion device.

2. The implant according to claim 1 wherein said implant comprises substantially allograft bone, autograft bone, xenograft bone, cancellous bone, cortical bone, a bone-like substance, a biocompatible synthetic material, and combinations thereof.

3. The implant according to claim 2 wherein said implant further comprises a notch, circular, hexagonal or other shaped protrusion, indenture, or cannulation for receiving a means to drive said implant into an implant location.

4. The implant according to claim 2 wherein said implant further comprises an external feature defined on a circumferential surface of said conical implant, wherein said feature is selected from the group consisting of a thread, grooves, and barbs.

5. The implant according to claim 1 wherein said second diameter is between about five-fold larger than said first diameter and equal to said first diameter.

6. The implant according to claim 3 wherein said implant comprises a cannulation extending partially from said back end to said front end or extending completely through said back end to said front end.

7. The implant of claim 1 wherein said at least one slot can be filled with autogenous bone, allogenic bone, xenograft bone, demineralized bone, bone paste, cellular material, growth factors, or stem cells.

8. The implant of claim 1 wherein said implant contains holes radiating from said slot to the exterior of said implant.

9. The implant according to claim 1 wherein the length of said implant ranges between about 1 mm to about 60 mm.

10. The conical implant according to claim 1 wherein the length of said slot ranges between about 0.25 mm to about 15 mm and the width of said slot ranges between about 0.1 mm to about 8 mm.

11. The conical implant according to claim 3 wherein the width of said notch ranges between about 0.1 mm to about 8 mm and the depth of said notch ranges between about 0.1 mm to about 6 mm.

12. The conical implant according to claim 6 wherein the diameter of said cannulation is between about 0.1 mm and about 4 mm.

13. The conical implant according to claim 4 wherein said thread pitch varies between about 0.5 mm to about 3 mm.

14. A method for bone osteotomy which comprises the steps of:
(a) forming a fracture or transverse incision in a bone in need of an osteotomy procedure;
(b) distracting said fracture with a distraction device;
(c) inserting within said fracture an implant comprising a front end of a first diameter, a rear end of a second diameter, wherein said first diameter is smaller than said second diameter, at least one slot formed transversely through a portion of said implant, and a means for attaching said implant to an implant insertion device; and
(d) removing said distraction device, leaving said implant in place.

15. The method according to claim 14 wherein said implant is composed substantially of bone, a bone-like substance, or a biocompatible synthetic material.

16. The method according to claim 15 wherein said implant further comprises a notch for receiving a means to drive said implant into an implant location.

17. The method according to claim 15 wherein said implant further comprises an external feature about the circumference of said conical implant, wherein said feature is selected from the group consisting of a thread, grooves, and barbs.

18. The method according to claim 14 wherein said second diameter is between about five-fold larger to about equal to said first diameter.

19. The method according to claim 16 wherein said implant comprises a cannulation extending partially from said back end to said front end or extending completely through said back end to said front end.

20. The method according to claim 14 wherein said at least one slot isfilled with autogenous bone, allogenic bone, xenograft bone, demineralized bone, bone paste, cellular material, stem cells, and growth factors.

21. The method according to claim 14 wherein said implant contains holes radiating from said slot to the exterior of said implant.

22. The method according to claim 14 wherein the length of said implant ranges between about 1 mm to about 60 mm.

23. The method according to claim 14 wherein the length of said slot ranges between about 0.25 mm to about 15 mm and the width of said slot ranges between about 0.1 mm to about 8 mm.

24. The method according to claim 16 wherein the width of said notch ranges between about 0.1 mm to about 8 mm and the depth of said notch ranges between about 0.1 mm to about 6 mm.

25. The method according to claim 19 wherein the diameter of said cannulation varies between about 0.1 mm and about 4 mm.

26. The method according to claim 17 wherein said thread pitch varies between about 0.5 mm to about 3 mm.

27. A method of making a conical implant for insertion into a bone in an osteotomy procedure which comprises the steps of:
(a) making a bone blank in the form of a cylinder;
(b) machining said bone blank in a lathe to define a first smaller diameter at a front end of said implant and a second larger diameter at a back end of said implant;
(c) machining said bone blank to define at least one slot through the body of said implant;
(d) drilling said bone blank to define holes radiating from the at least one slot to the external surface of said implant;
(e) driving said bone blank through a cutter assembly for machining an external feature into the circumference of said implant;
(f) machining an instrument attachment cannulation into the center of said second larger diameter through the back end of said implant;
(g) machining a notch through the diameter of said second larger end for receiving a means to drive said implant into a fracture site; and
(h) disinfecting said implant.

28. The method according to claim 27 wherein said implant is composed substantially of bone, a bone-like substance, or a biocompatible synthetic material.

29. The method according to claim 28 wherein a notch for receiving a means to drive said implant into an implant location is formed in said implant.

30. The method according to claim 28 further comprising machining an external feature about the circumference of said conical implant, wherein said feature is selected from the group consisting of a thread, grooves, and barbs.

31. The method according to claim 27 further comprising forming said second diameter to between about five-fold larger than to about equal to said first diameter.

32. The method according to claim 29 further comprising forming a cannulation extending partially from said back end to said front end or extending completely through said back end to said front end.

33. The method according to claim 27 further comprising filling said at least one slot with autogenous bone, allogenic bone, xenograft bone, demineralized bone, bone paste, cellular material, growth factors, stem cells or combinations thereof.

34. The method according to claim 27 further comprising forming holes radiating from said at least one slot to the exterior of said implant.

35. The method according to claim 27 comprising forming the length of said implant to between about 1 mm to about 60 mm.

36. The method according to claim 27 comprising forming the length of said at least one slot to between about 0.25 mm to about 15 mm and the width of said at least one slot to between about 0.1 mm to about 8 mm.

37. The method according to claim 29 further comprising forming the width of said notch to between about 0.1 mm to about 8 mm and the depth of said notch to between about 0.1 mm to about 6 mm.

38. The method according to claim 32 comprising forming the diameter of said cannulation to between about 0.1 mm and about 4 mm.

39. The method according to claim 30 comprising forming the thread pitch to between about 0.5 mm to about 3 mm.
